(19) United States
(12) Patent Application Publication
(10) Pub. No.: US 2018/0116866 A1
(43) Pub. Date: May 3, 2018

(54) EYE PAD HEATER

(71) Applicant: William Agnew, Salisbury, MD (US)

(72) Inventor: William Agnew, Salisbury, MD (US)

(21) Appl. No.: 15/341,365

(22) Filed: Nov. 2, 2016

(51) Int. Cl. A61F 7/02

(52) U.S. Cl. CPC A61F 7/0241; A61F 2007/0004; A61F 7/02

(57) ABSTRACT

An eye pad heater is provided. The eye pad heater includes a housing with at least one slot leading into an internal compartment. A heating element is disposed within the housing. The heating element produces heat within the internal compartment up to about 35 degrees Celsius. A power source provides power to the heating element. A switch is operable to open and close a circuit between the power source and the heating element.

### BACKGROUND OF THE INVENTION

The present invention relates to eye treatment and, more particularly, to an eye pad heater.

The eyelids are important to ocular health because they protect the eyes from airborne contaminants, such as pollen, dust particles or other foreign bodies. The eyelids contain several glands including the lacrimal glands and the specialized form of the sebaceous glands, the meibomian glands, which produce layers of tear film that are critical for healthy eyes.

The eyes and eyelids are subject to problems like blepharitis, dry eyes and inflammation of the meibomian glands. Ocular drug delivery includes the delivery of medications to the eye to treat an assortment of eye diseases. A common method of therapy for dry eye is the heated eye compress. Homemade eye compresses are currently recommended, usually a cloth or paper towel heated with hot tap water, to be applied to the eyes daily. These are unsafe due to lack of control over sterility and temperature control. Sterile eye compresses exist, but there is currently no apparatus to safely heat them to a consistent temperature.

As can be seen, there is a need for an apparatus that heats sterile eye compresses, hereafter referred to as “eye pads” to a proper temperature.

### SUMMARY OF THE INVENTION

In one aspect of the present invention, an eye pad heater comprises: a housing comprising at least one slot leading into an internal compartment; a heating element disposed within the housing, wherein the heating element produces heat within the internal compartment up to about 30 degrees Celsius; a power source providing power to the heating element; and a switch operable to open and close a circuit between the power source and the heating element.

In another aspect of the present invention, a method of warming eye pads comprises: providing an eye pad heater comprising: a housing comprising at least one slot leading into an internal compartment; a heating element disposed within the housing, wherein the heating element produces heat within the internal compartment up to about 30 degrees Celsius; a power source providing power to the heating element; and a switch operable to open and close a circuit between the power source and the heating element; saturating an eye pad in a solution; placing the eye pad through the slot and into the internal compartment; and activating the switch to close the circuit.

These and other features, aspects and advantages of the present invention will become better understood with reference to the following drawings, description and claims.

### BRIEF DESCRIPTION OF THE DRAWINGS

FIG. 1 is a perspective view of an embodiment of the present invention;

FIG. 2 is a cutaway detail view taken along line 2-2 in FIG. 1; and

FIG. 3 is the cutaway detail view of FIG. 2 showing the embodiment of the present invention in use.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

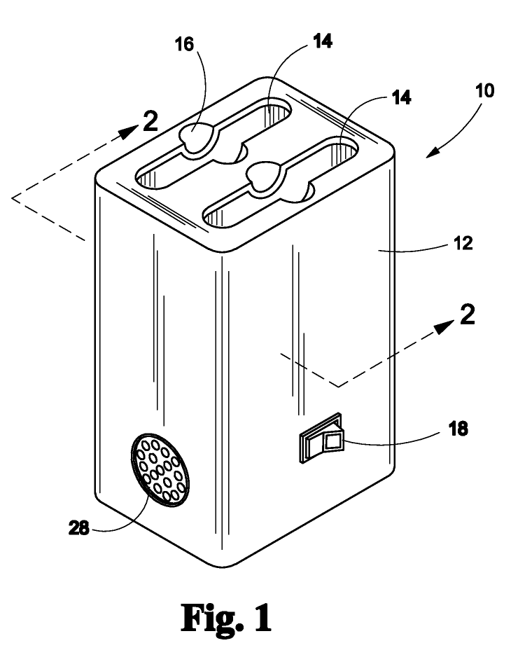

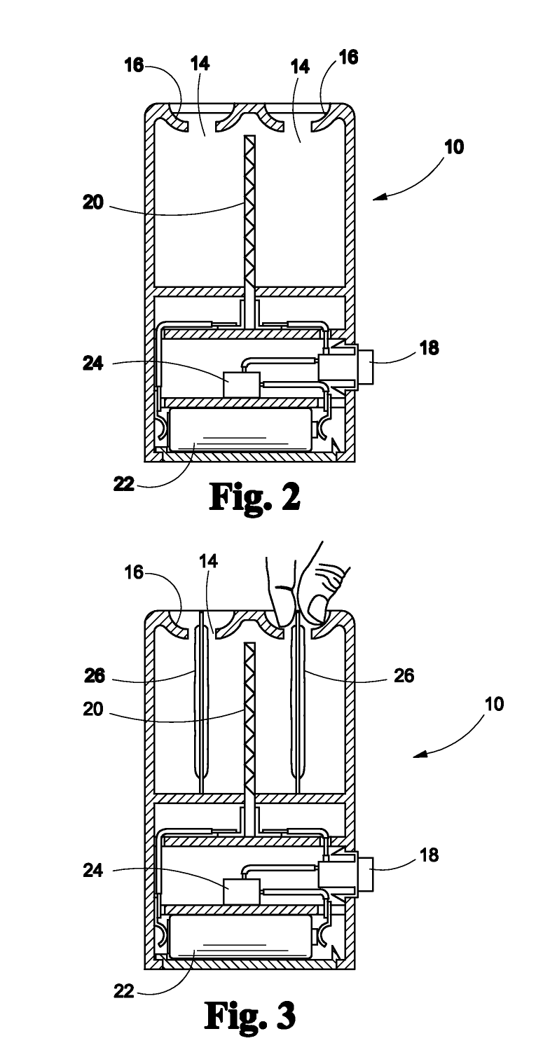

Referring to FIGS. 1 through 3, the present invention includes an eye pad heater 10. The eye pad heater 10 includes a housing 12 with at least one slot 14 leading into an internal compartment. A heating element 20 is disposed within the housing 12. The heating element 20 produces heat within the internal compartment up to about 30 degrees Celsius. A power source 22 provides power to the heating element 20. A switch 18 is operable to open and close a circuit between the power source 22 and the heating element 20.

The housing 12 of the present invention includes a top wall, a bottom wall and a sidewall. The at least one slot 14 is formed on the top wall and the bottom wall supports the eye pad heater 10 in an upright position. The housing 12 may be made of a solid material, such as a solid plastic or metal. The housing 12 may be about 2-5 inches tall and about 1-4 inches wide. For example, the housing 12 may be about 3 inches tall and 2 inches wide.

The at least one slot 14 may include a first slot 14 and a second slot 14, each leading into the internal compartment. Therefore, a user may heat up two eye pads 26 at one time. In certain embodiments, the housing 12 may include more than two slots 14, such as four slots 14 or eight slots 14. The slots 14 are elongated and are formed by an inner edge having a first end, a second end and an elongated first side and second side. The slots 14 are shaped to receive the eye pads 26 and may be about an inch and a half long and about a quarter of an inch wide. Each of the slots 14 may include a pair of notches 16. The notches 16 are formed in the elongated first side and second side respectively. The notches 16 allow a user to easily remove the eye pad 16 from the internal compartment, since a top edge of the eye pad 26 is disposed in between the notches 16.

The heating element 20 may include a metal coil, a ceramic, a laser, a light emitting diode, plasma, chemical heating or the like. In certain embodiments, the heating element 20 produces heat within the internal compartment from about 25 degrees Celsius up to about 35 degrees Celsius. For example, the heating element 20 produces heat within the internal compartment at about 30 degrees Celsius. Therefore, a user may insert an eye pad 26 within the internal compartment and heat the eye pad 26 to the optimal temperature, preventing burning the eyelid due to overheating.

As illustrated in the Figures, the present invention includes a circuit connecting the power source 22 to the heating element 20. The switch 18 is operable to open and close the circuit, and thereby turn on and off the heating element 20. In certain embodiments, the switch 18 is protruding from the sidewall. The switch may include a rocket switch, a toggle switch, an on and off button and the like.

A temperature gauge 24 may also be connected to the circuit. In such embodiments, the temperature gauge 24 is disposed within the housing 20 and measures temperature within the internal compartment. Once the temperature gauge 24 measures a threshold temperature, the circuit may automatically open to turn the heating element 20 off. The temperature gauge 24 thereby prevents the internal compartment from exceeding a threshold temperature. The threshold temperature may be from about 25 degrees Celsius up to about 35 degrees Celsius, such as about 30 degrees Celsius.

The eye pad heater 10 may further include an indicator 28 operable to indicate that the circuit has been opened due to the temperature gauge 24. The indicator 28 produces at least one of a sound and a light. Therefore, when the temperature gauge 24 opens the circuit, the indicator 28 produces the sound or light to notify the user that the eye pad 26 has been sufficiently heated.

In certain embodiments, the power source 22 may be a battery disposed within the housing 12. The battery may be a AA battery, AAA battery, a watch battery and the like. The power source may also include A/C or D/C power. For example, a cord and a plug may run from the housing 12 and may be plugged into a power outlet. In certain embodiments, the present invention may include a rechargeable battery. In such embodiments, the housing may include a USB port connected to the rechargeable battery. A USB cord may connect the rechargeable battery to an outside power source to recharge.

The eye pad 26 of the present invention may be an absorbent cotton fiber pad 26. The absorbent cotton fiber pad 26 may be stored within a packaging so that the absorbent cotton fiber pad 26 remains sterile while not in use. The absorbent cotton fiber pad 26 may be removed from the packaging prior to use. Once the absorbent cotton fiber pad 26 has been removed, a liquid medication or a saline solution may be applied to the pad 26. The absorbent cotton fiber pad 26 may absorb and retain the medication or saline solution within the cotton fibers. The eye pad 26 may then be inserted into the internal compartment through the slots 14 and heated by the eye pad heater 10.

A method of warming eye pads 26 may include the following. The eye pad heater 10 disclosed above may be provided. The eye pad 26 is then saturated with a solution. The eye pad 26 is placed through the slot 14 and into the internal compartment. The switch 18 is then activated to close the circuit and power the heating element 20. The eye pad heater 10 may further include an indicator 28 operable to indicate that the circuit is open. The indicator 28 produces at least one of a sound and a light. The method may further include the step of removing the eye pad 26 from the internal compartment once the indicator 28 produces the indication.

It should be understood, of course, that the foregoing relates to exemplary embodiments of the invention and that modifications may be made without departing from the spirit and scope of the invention as set forth in the following claims.

What is claimed is:

1. An eye pad heater comprising:
a housing comprising at least one slot leading into an internal compartment;
a heating element disposed within the housing, wherein the heating element produces heat within the internal compartment up to about 35 degrees Celsius;
a power source providing power to the heating element; and
a switch operable to open and close a circuit between the power source and the heating element.

2. The eye pad heater of claim 1, wherein the housing comprises a top wall, a bottom wall and a sidewall, wherein the at least one slot is formed on the top wall and the bottom wall supports the eye pad heater in an upright position.

3. The eye pad heater of claim 2, wherein the at least one slot comprises a first slot and a second slot, each leading into the internal compartment.

4. The eye pad heater of claim 2, wherein the at least one slot is an elongated slot formed by an inner edge having a first end, a second end and an elongated first side and second side.

5. The eye pad heater of claim 4, further comprising a pair of notches each formed in the elongated first side and second side respectively.

6. The eye pad heater of claim 2, wherein the switch is protruding from the sidewall.

7. The eye pad heater of claim 1, wherein the heating element produces heat within the internal compartment from about 25 degrees Celsius up to about 35 degrees Celsius.

8. The eye pad heater of claim 7, wherein the heating element produces heat within the internal compartment at about 30 degrees Celsius.

9. The eye pad heater of claim 1, further comprising a temperature gauge disposed within the housing and operable to open the circuit once the temperature gauge reaches threshold temperature.

10. The eye pad heater of claim 9, wherein the threshold temperature is from about 25 degrees Celsius up to about 35 degrees Celsius.

11. The eye pad heater of claim 1, wherein the power source is a battery.

12. A method of warming eye pads comprises:
providing an eye pad heater comprising:
a housing comprising at least one slot leading into an internal compartment;
a heating element disposed within the housing, wherein the heating element produces heat within the internal compartment up to about 35 degrees Celsius;
a power source providing power to the heating element; and
a switch operable to open and close a circuit between the power source and the heating element;
saturating an eye pad in a solution;
placing the eye pad through the slot and into the internal compartment; and
activating the switch to close the circuit.

13. The method of claim 12, wherein the eye pad heater further comprises a temperature gauge disposed within the housing and operable to open the circuit once the temperature gauge reaches 35 degrees Celsius.

14. The method of claim 13, further comprising an indicator operable to indicate that the circuit is open, wherein the indicator produces at least one of a sound and a light.

15. The method of claim 14, further comprising the step of removing the eye pad from the internal compartment once the indicator produces the indication.
